(19) United States
(12) Patent Application Publication
(10) Pub. No.: US 2018/0214292 A1
(43) Pub. Date: Aug. 2, 2018

(54) Intragastric device, comprising a rounded element at a distal end and an EXPANDABLE duodenal anchor, and methods of using the same

(71) Applicants: National University of Singapore, Singapore (SG); Allium Medical Ltd, Caesarea (IL)

(72) Inventors: Yeela Doron, Tel Aviv (IL); Gonen Somekh, Kerem Maharal (IL); Ziv Kalfon, Ein Hod (IL)

(21) Appl. No.: 15/747,440

(22) PCT Filed: Aug. 7, 2016
(86) PCT No.: PCT/IL2016/050862

§ 371 Date: Feb. 16, 2018

Related U.S. Application Data
(60) Provisional application No. 62/205,761, filed on Aug. 17, 2015.
(60) Provisional application No. 62/274,397, filed on Jan. 4, 2016.

(51) Int. Cl. A61F 5/00

(52) U.S. Cl. CPC A61F 5/0079

(57) ABSTRACT

Disclosed is an intragastric device (200) comprising a sleeve (501) including a pyloric sleeve region (205) and a duodenal sleeve region (209), a rounded element (405) at a distal end of the duodenal sleeve region (209) and an expandable duodenal anchor (207), which is positioned between the pyloric sleeve region (205) and the duodenal sleeve region (209).

### FIELD OF THE INVENTION

This invention is directed to an intragastric device, comprising a rounded element at a distal end and an expandable duodenal anchor, and methods of using the same.

### BACKGROUND OF THE INVENTION

One of the major comorbidities related to obesity is Type II Diabetes Mellitus (T2DM), wherein roughly 85% of obese adults suffer from this infliction. Nowadays, bariatric procedures are the most common treatments for obese patient who also suffer from uncontrolled T2DM. The acute glucose-lowering effect of certain bariatric procedures before any significant weight loss has occurred has been known for decades. Type 2 diabetes remission rates after undergoing Roux-en-Y gastric bypass (RYGB), which is the most common bariatric procedure, were reported to be 80%. Importantly, bariatric surgery seems to improve several components of the metabolic syndrome, and type 2 diabetes specific mortality rates have been demonstrated to be up to 90% lower in RYGB-treated subjects compared with non-treated subjects.

RYGB includes surgical formation of a small stomach pouch. The small intestine is divided distally from the ligament of Treitz, and the distal end is connected to the newly formed small stomach pouch (gastro-jejunostomy). The upper part of the small intestine is then reattached in a Y-shaped configuration distally to the gastrojejunostomy, thus forming a “secretory limb” where gastric juices, bile, and pancreatic exocrine products enter duodenum and flow onward to the jejunojejunostomy. In contrast, nutrients pass directly from the small stomach pouch into the “alimentary limb” (distal jejunum). In this way, the undigested food is mixed with bile and pancreatic and gastric juices distally to the duodenum.

As a minimally invasive alternative for the RYGB procedure, a gastro-duodenal sleeve can be introduced. The sleeve, a flexible tubular envelop which is inserted endoscopically and placed in the proximal small intestine, creates an internal bypass in the small intestine, thus mimicking the effects of Roux-en-Y bariatric surgery—without surgery. In effect, the sleeve functions as a barrier that prevents chyme from mixing with the gastric juices.

Despite many advances in the design of intragastric diabetes treatment devices, there remains a need for an improved intragastric device comprising a gastro-duodenal sleeve that address certain drawbacks of previous devices or otherwise can be implanted for longer periods than before.

### SUMMARY OF THE INVENTION

Embodiments of the invention are directed to an intragastric device (200) comprising a sleeve (501) including a pyloric sleeve region (205) and a duodenal sleeve region (209), a rounded element (405) at a distal end of the duodenal sleeve region (209) and an expandable duodenal anchor (207), which is positioned between the pyloric sleeve region (205) and the duodenal sleeve region (209).

According to some embodiments, the sleeve (501) further comprises one or more additional anchors. According to further embodiments, the sleeve (501) is axially collapsible. According to further embodiments, the sleeve (501) includes reinforcements. According to further embodiments, the sleeve (501) resists structural degradation for at least six months within the gastrointestinal tract. According to further embodiments, the sleeve (501) further comprises a gastric anchor (203). According to further embodiments, only a distal end of the duodenal anchor (207) is attached to the sleeve (501). According to further embodiments, none of the anchors include tissue piercing elements. According to further embodiments, at least one of the anchors is prepared from a nitinol structure compressed to be delivered transorally, and expandable for being in contact with at least part of antrum area of the stomach or the intestine, depending on the position of the anchor. According to further embodiments, the duodenal anchor (207) and the gastric anchor (203) are coupled by a coupling element (1002). According to further embodiments, the gastric anchor (203) is prepared as an opened shape, wherein at least two ends (1203, 1205) of the gastric anchor (203) are connected to one another only by a polymeric material. According to further embodiments, the polymeric material is the polymeric material of the sleeve (501). According to further embodiments, at least one of the ends (1205) of the gastric anchor (203) is coupled to the duodenal anchor (207) at a proximal end of the duodenal anchor (207).

Some embodiments are directed to the intragastric device (200) as detailed herein for use in treating type II diabetes, small intestine perforation, small intestine abscess, obesity, or any combination thereof.

Additional embodiments are directed to a method of deploying the intragastric device (200) as detailed herein, wherein the method comprises:

delivering the device (200) folded within a capsule (401) through the mouth, wherein the capsule (401) is attached to an outer tube (403);

positioning the capsule (401) in the pyloric sphincter (105);

using an inner tube (503) connected to a rounded element (405) to push and thereby deploy the sleeve (501) from the capsule (401) positioned in the pyloric sphincter (105) along a predefined length of the small intestine (107), thereby releasing the device (200) from the capsule (401), thus causing the device (200) to be transformed from a compacted configuration to an expanded configuration, wherein the duodenal anchor (207) is positioned on the duodenal side of the pyloric sphincter (105);

removing the inner tube (503); and

pulling the outer tube (403) out of the mouth.

According to some embodiments, the rounded element (405) has a folded configuration and an unfolded configuration and wherein the rounded element (405) is in the folded configuration when connected to the inner tube (503) and is further transformed to the unfolded configuration when the inner tube (503) is removed.

Further embodiments are directed to a method of removing the intragastric device (200) as detailed herein from a patient's body, wherein the method comprises:

introducing a withdrawing device (1210) into the patient's stomach (101), wherein the withdrawing device (1210) comprises an outer sheath (1206), an inner sheath (1204), positioned inside the outer sheath (1206), and a grasping element (1202) extending from the distal end of the inner sheath (1204);

grasping an end (1203) of the gastric anchor (203) that is not directly attached to the coupling element (1002);

pulling the inner sheath (1204) in a proximal direction until the gastric anchor (203) is pulled into the outer sheath (1206) and the sleeve (501) is torn distally to where the gastric anchor (203) was attached to the sleeve (501), such that a coupling element (1002) provides a connection between the duodenal anchor (207) and the withdrawing device (1210);

positioning a distal end of the outer sheath (1206) in the pyloric sphincter (105);

further pulling the inner sheath (1204) in the proximal direction, which pulls the coupling element (1002), causing the duodenal anchor (207) to contract and to be pulled into the outer sheath (1206); and

pulling the outer sheath (1206) in a proximal direction from the pyloric sphincter (105) through the stomach (101) and out of the patient's body.

Further embodiments are directed to a withdrawing device (1210) for withdrawing an intragastric device comprising an outer sheath (1206), an inner sheath (1204) positioned inside the outer sheath (1206), and a grasping element (1202) extending from the distal end of the inner sheath (1204).

Further embodiments are directed to a kit comprising the intragastric device (200) as detailed herein and the withdrawing device (1210) as detailed herein.

### BRIEF DESCRIPTION OF THE DRAWINGS

Embodiments of the invention will be understood and appreciated more fully from the following detailed description in conjunction with the figures, which are not to scale, in which like reference numerals indicate corresponding, analogous or similar elements, and in which:

FIG. 1 is a cross sectional view of a portion of the digestive tract;

FIG. 2 is perspective view of an embodiment of part of the intragastric device according to the invention;

FIG. 3 is a cross sectional view of an embodiment of the intragastric device after deployed in the digestive tract;

FIG. 4 is a cross sectional view of a portion of the digestive tract at the beginning of the deployment of an embodiment of the intragastric device, presenting the outer tube, the rounded element and the device's position in the digestive tract;

FIG. 5A and FIG. 5B, collectively referred to as FIG. 5, are cross sectional views of a portion of the digestive tract during the deployment of an embodiment of the intragastric device, presenting the outer tube, the inner tube and the device's position in the digestive tract as well as the distal end of the intragastric device with the rounded element at its end;

FIG. 6 is a cross sectional view of an embodiment of the intragastric device compacted within the capsule;

FIGS. 7A and 7B present a perspective view of an embodiment of the rounded element, wherein FIG. 7A presents the rounded element in a folded configuration while FIG. 7B presents the rounded element in an unfolded configuration;

FIG. 8 is a perspective view of an embodiment of the rounded element, folded and attached to the inner tube;

FIG. 9 is a cross sectional view of an embodiment of the rounded element, folded and attached to the inner tube;

FIGS. 10A and 10B present proximal views of an embodiment of an expandable anchor, e.g., the duodenal anchor, that is attached to the sleeve only at its distal end, wherein FIG. 10A presents the shape of the anchor when no tension is applied to the sleeve, while FIG. 10B presents the shape of the anchor, when proximal tension is applied on the sleeve;

FIGS. 11A and 11B present a proximal view of an embodiment of part of the intragastric element, wherein FIG. 11A presents a view before pulling on the coupling element while FIG. 11B presents a view after pulling on the coupling element has begun;

FIGS. 12A-12F present cross sectional views of embodiments of the method of removing an embodiment of the intragastric sleeve from the patient's body, wherein FIG. 12A is a cross sectional view of the intragastric sleeve and the withdrawal device, before they come in contact; FIG. 12B is a cross sectional view presenting the grasper grasping the gastric anchor; FIG. 12C is a cross sectional view of the withdrawing means and the intragastric device after the gastric anchor has been folded into the outer sheath and detached from the sleeve; FIG. 12D is a cross sectional view of the withdrawing means positioned in the pyloric sphincter; FIG. 12E is a cross sectional view of the withdrawing means when the duodenal anchor is partially collapsed and begins to enter the outer sheath; and FIG. 12F is a cross sectional view of the withdrawal means pulled back into the stomach after both anchors have passed through the distal end of the outer sheath.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Embodiments of the invention are directed to an intragastric device, comprising a sleeve including a pyloric sleeve region and a duodenal sleeve region, a rounded element at a distal end of the duodenal sleeve region and an expandable duodenal anchor, which is positioned between the pyloric sleeve region and the duodenal sleeve region, such that those regions are essentially defined by the position of the duodenal anchor. According to some embodiments, the intragastric device is used for treating type II diabetes, obesity, small intestine perforation, small intestine abscess or any combination thereof.

According to some embodiments, the sleeve has a tubular shape and includes a pyloric sleeve region and a duodenal sleeve region. The pyloric sleeve region is defined as the region of the sleeve stretching from the duodenal anchor towards the stomach and passing through the pyloric sphincter, while the duodenal sleeve region is defined as the region of the sleeve stretching from the duodenal anchor into the duodenum.

The sleeve may be axially collapsible. According to some embodiment, the sleeve is formed from a polymeric material as polyurethane, polyethylene, silicon, PTFE, not coated or coated with parylene. According to some embodiments, the sleeve is reinforced, e.g., with nitinol or with polymer such as polyurethane, polyethylene, silicon, PTFE or with a composite material or by variation in the sleeve polymer thickness. According to some embodiments the reinforcements have circular (ring) shape or shape of a spiral located along the sleeve. According to some embodiments, the reinforcements are placed in any desired locations in the sleeve. According to some embodiments the reinforcement could be at any angle to the axis of the sleeve. According to some embodiments, the reinforcements may be integrated into the polymeric material of the sleeve. The reinforcements are sufficiently strong, such that when the sleeve is expanded the reinforcements may line the inner intestine wall and apply certain pressure to the intestine walls to keep the sleeve always open.

According to some embodiments, the material from which the sleeve is formed resists structural degradation within the gastrointestinal tract over a predefined period of time. According to some embodiments the predefined period of time is about one month, two months, four months, six months, eight months, a year, two years or more. According to some embodiments, the predefined period of time is at least about six months.

Once the sleeve is placed in the intestine it limits nutrient absorption and prevents chyme from mixing with the biliopancreatic juices while passing through the sleeve. The sleeve also assists in closing fistula, if such exists, since the sleeve prevents contact of chyme with the damaged tissue and stimulates faster damaged tissue recovery.

According to some embodiments, the sleeve includes at least two radially collapsible anchors, each having a collapsed configuration and an expanded configuration. The expanded configuration of the anchors prevents the passage of the device through the pyloric sphincter, wherein at least one of the anchors (related to herein as the “gastric anchor”) is placed on the stomach side of the pyloric sphincter and at least one anchor (related to herein as the “duodenal anchor”) is placed on the duodenal side of the pyloric sphincter. Thus, when expanded, the size of each of those anchors is larger than the pyloric sphincter. When in the collapsed configuration, the device may be delivered with ease into the intestine and at least partially through the pyloric sphincter.

The anchors may be concentrically disposed around the sleeve. The anchors may also be held away from one another so as to impede movement of the anchored sleeve in both a proximal and a distal direction. According to some embodiments, any two or more anchors may be coupled to one another by connectors, e.g., prepared from nitinol or polymeric materials.

According to some embodiments, the sleeve includes two anchors, a gastric anchor and a duodenal anchor, which, as detailed above, are positioned such that the gastric anchor is on the stomach side of the pyloric sphincter, while the duodenal anchor is positioned on the duodenal side of the pyloric sphincter.

According to some embodiments, the gastric anchor comprises a funnel shaped structure that extends into the stomach, wherein the sleeve widens in the stomach in a proximal direction along the funnel shaped structure. When expanded the funnel shaped structure may line the inner stomach wall and applies certain pressure to the stomach walls.

According to some embodiments, the duodenal anchor is fully integrated within the sleeve. According to other embodiments, the duodenal anchor is expandable. According to some embodiments, the movement of the duodenal anchor is obtained by a structure in which only the distal end of the duodenal anchor is attached to the sleeve. If only the distal end of the duodenal anchor is attached to the sleeve, the resistance of the sleeve to proximal movement is improved. When proximal tension is applied on the sleeve, the attached distal end of the duodenal anchor will be narrowed and cause the expansion of anchor's proximal end, thus preventing its passage through the pylorus.

According to some embodiments, the gastric anchor does not include tissue piercing elements. According to some embodiments, the duodenal anchor does not include tissue piercing elements. According to some embodiments, none of the anchors include tissue piercing elements. According to some embodiments, at least one of the anchors does not include tissue piercing elements.

According to some embodiments any one or more of the anchors is formed by a nitinol structure sufficiently compressible to be delivered transorally, and sufficiently large when expanded to contact with the antrum area of the stomach or the intestine, depending on the position of the anchor. When in the expanded configuration, any one or more of the anchors may include a central lumen that defines the flow orifice from the stomach and through the sleeve. According to some embodiments, any one of the anchors may be in a rounded shape having any number of finger like projections, as shown, for example, in the figures. When expanded or opened the finger like projections may lie against the stomach walls, applying a certain pressure thereto.

According to other embodiments, any one or more of the anchors may comprise, be coupled to or be adjacent to, an inflatable balloon. During the deployment of the device the balloon may be deflated to a delivery size profile and when the sleeve is positioned as desired, the balloon may be inflated to its deployed size. The profile of the inflated balloon is larger than the diameter of the pyloric sphincter (when opened), to prevent distal migration of the balloon into the duodenum. The volume of the balloon may be adjusted in order to maximize or minimize its movement in the stomach or duodenum and as a result to control the amount of movement of the sleeve. The balloon's shape may be predefined as desired, including, though not limited to, a ring, two or more rings connected to one another or any other shape to comply with the stomach or intestine walls.

According to some embodiments, any one or more of the anchors may include both a nitinol structure and an inflatable balloon. Thus, even if the balloon is ruptured, the anchor will remain in place.

According to some embodiments, any two anchors, e.g., the gastric anchor and the duodenal anchor, are connected to one another by a polymeric material. According to some embodiments, the polymeric material is the polymeric material of the sleeve. According to some embodiments, no additional connections are provided between the two anchors. According to other embodiments, any two anchors, e.g., the duodenal anchor and the gastric anchor may be rigidly connected to one another to effectively form one trans-pyloric anchor. The trans-pyloric anchor will prevent movement of the sleeve both in the distal and in the proximal directions. The rigid connection of the two anchors, e.g., the duodenal and gastric anchor, may comprise of a channel that chyme may pass through. The diameter of the channel may be slightly smaller than the diameter of the pyloric sphincter (when opened). This channel may practically prevent the pyloric sphincter from closing. According to some embodiments, the connectors include several members that form a bundle, wherein the bundle is concentrically aligned with the pyloric sphincter and allows the sphincter to close around it.

According to some embodiments, any one of the anchors, including the gastric anchor, may be prepared such that it forms an opened structure, wherein at the opening, at least two ends of the structure are connected to one another only by the polymeric material of the sleeve. According to other embodiments, any one of the anchors, including the gastric anchor, may be prepared as structure having both closed and opened configurations. According to some embodiments, the main body of the anchor is prepared as an opened shape, e.g., an opened circle, ellipse, or any other shape that may anchor the sleeve in place without damaging the body's tissue. The shape of the anchor may be symmetrical or asymmetrical. The opened shape may be closed by any appropriate connecting means, such as a wire, twine, catheter and the like, that may pass through, e.g., holes or hooks, at the two ends of the opened shape, thereby holding those two ends together in the closed configuration. Once the connecting means are removed, the anchor may be transformed from the closed to the opened configuration, wherein in the opened configuration, the two ends of the anchor are held together, or in the vicinity of one another, only by the polymeric material of the sleeve, i.e., no nitinol structure connects the two ends. According to some embodiments, any one of the anchors, e.g., the gastric anchor, includes two such opening, wherein the structure includes at least four ends, each two pairs of ends being connected to one another only by the polymeric material of the sleeve.

According to some embodiments, any two anchors, e.g., the gastric and the duodenal anchor are coupled to one another by any appropriate coupling means. According to some embodiments, the coupling means couple one end of an anchor, e.g., the gastric anchor, having an opened shape, as detailed above, with a second anchor, e.g., the duodenal anchor, wherein the coupling means stretch axially along the length of the sleeve between the two anchors. According to further embodiments, the coupling means connect both ends of the opened structure anchor to one another and then stretch along the length of the sleeve to couple the two anchors to one another.

According to some embodiments, when the intragastric device is to be removed from the patient's body, it is coupled to withdrawal means. According to some embodiments, the withdrawal means comprises an outer sheath for storing the anchors, an inner sheath positioned within the outer sheath and a grasping element that may extend from the distal end of the inner sheath. According to some embodiments, the grasping element includes teeth, hooks, or the like, in order to enable it to grasp/attach to other elements.

According to some embodiments, the intragastric device includes coupling means, as detailed above, coupling two anchors, e.g., the gastric and the duodenal anchor, to one another. When desired to withdraw the intragastric device from the patient's body, the withdrawal means is inserted into the stomach, wherein the grasping element may grasp the gastric anchor, thereby causing the gastric anchor to collapse and/or be perforated and be pulled into the outer sheath, e.g., by pulling the inner sheath in the proximal direction. According to some embodiments, the grasping element grasps a part of the gastric anchor that is not directly attached to the coupling element. According to some embodiments, the grasping element grasps a part of the gastric anchor that is opposite where the gastric anchor is attached to the coupling element. According to some embodiments, pulling and collapsing the gastric anchor into the outer sheath causes the sleeve to be torn distally to where the gastric anchor was attached, such that the coupling element provides the connection between the remaining part of the sleeve, including the duodenal anchor, and the withdrawal means. As detailed herein, collapsing/folding an anchor into the outer sheath is meant to include, e.g., tearing it away from the sleeve, such that it assumes a more linear shape that can fit into the sheath, which may also be related as extending or pulling the anchor into the sheath.

According to some embodiments, the gastric anchor has an opened structure and is coupled to the duodenal anchor via a coupling element attached to one end of the opened structure of the gastric anchor, from where it stretches axially along the sleeve and is attached to the duodenal anchor, such that when the grasping element grasps a part of the gastric anchor that is opposite to where the gastric anchor is attached to the coupling element, the gastric anchor is easily collapsed and folded (or torn away from the sleeve, such that it assumes a more linear shape, such that it is extended or pulled) into the outer sheath.

Once the gastric anchor is collapsed and folded (or extended or pulled) into the outer sheath, the distal end of the outer sheath may then be positioned in the pyloric sphincter. The inner sheath may then be pulled further in the proximal direction, thereby pulling the coupling means, which causes the proximal end of the duodenal anchor to collapse first, allowing it to be pulled into the outer sheath. According to some embodiments, the duodenal anchor is collapsed by the coupling means when the coupling means is pulled radially inward on the proximal end of the duodenal anchor. According to some embodiments, the coupling means is attached to the circumference of the duodenal anchor, such that pulling in the proximal direction on the coupling means causes the duodenal anchor to contract/collapse/be compacted.

The device may be deployed and/or withdrawn endoscopically through the mouth.

The device may be delivered into the intended location using a delivery system. The delivery system may comprise an outer catheter/tube for storing the device, a water channel and an inner catheter/tube that extends beyond the outer catheter/tube for deploying the device both in the stomach and along the small intestine through the pyloric sphincter.

To facilitate the deployment of the device in the small intestine, the inner catheter may be coupled to a rounded element. It is noted that the rounded element may be circular, elliptic, mostly rounded, including one or more kinks, symmetric or asymmetric, or any other possible shape that is atraumatic, i.e., not harmful, to the body's tissue during and after deployment of the device. According to some embodiments, the rounded element is a ring element that is integrated in the device and that facilitates its deployment in the intended location. The rounded element is positioned at the distal end of the duodenal sleeve region. The rounded element is sufficiently thick so as to function as an atraumatic end when advanced along the small intestine. According to some embodiments, the rounded element is between 1 and 4 mm thick and the diameter of the tube is between 20 to 30 mm. According to some embodiments, the rounded element (related to herein also as the “ring”, “rounded element”, “atraumatic element” or the “atraumatic ring element”) at the distal end of the sleeve is coupled to a catheter using a coupling wire. When advancing the catheter, the rounded element leads the sleeve through the small intestine without damaging the tissue. According to some embodiments, the rounded element is of sufficient size/volume to be carried downwards in the small intestine by the peristaltic waves and assist the deployment of the device. After completing the deployment, the inner catheter may be remotely detached from the rounded element. According to some embodiments, while the device is in the intestinal tract, the rounded element preserves the folded end opening at the distal end of the sleeve, such that the distal end of the sleeve is supported in a manner that keeps it opened.

According to further embodiments, the rounded element comprises two or more bodies at its distal edge that are integrated within the sleeve. The shape of the bodies may be a sphere, disk, ellipse or any other appropriate shape designed to be atraumatic, e.g., does not include sharp edges. While the sleeve is folded within the outer catheter, the bodies are held in close proximity to one another, thereby creating a mass of the combined bodies, i.e., a mass larger than each of the bodies on its own. This larger mass is coupled to the inner catheter and functions as an atraumatic end during sleeve deployment.

According to some embodiments, the rounded element is designed as a part of the sleeve. According to other embodiments, the rounded element is detachable from the sleeve and may be detached after deployment. Such a detachment may be actively initiated by a maneuver as part of the deployment procedure. According to other embodiments, the detachment may be obtained due to peristaltic movements of the small intestine. Passive detachment of the rounded element by peristalsis may be achieved by weakened joints, weakened connectors, a perforation line proximal to the rounded element or the like.

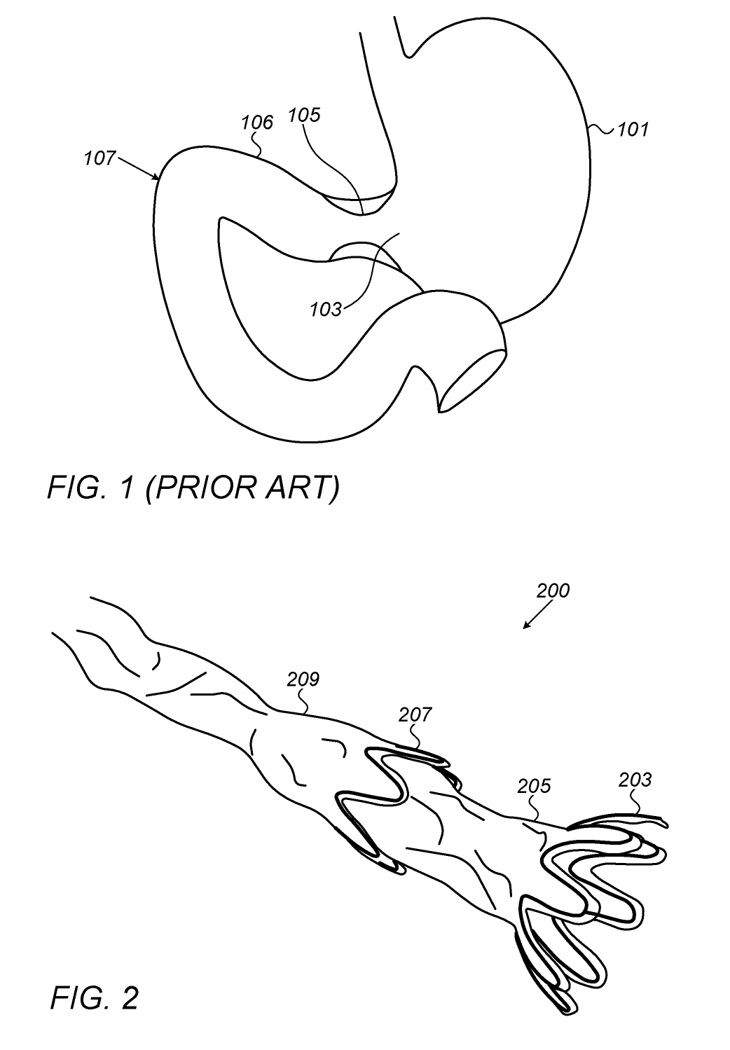

Reference is now made to FIG. 1, presenting a cross sectional view of a portion of the digestive tract (Prior Art). FIG. 1 presents a cross section of the stomach (101), the pylorus (103), the pyloric sphincter (105), the duodenal bulb (106) and the duodenum (107).

Reference is now made to FIG. 2, presenting a perspective view of an embodiment of part of the intragastric device (200). The presented embodiment includes two anchors, gastric anchor (203) and duodenal anchor (207). When placed in the digestive tract, the gastric anchor (203) is anchored on the stomach side of the pyloric sphincter, i.e., proximal to the pyloric sphincter, duodenal anchor (207) is anchored on the duodenal side of the pyloric sphincter, i.e. distally to the pyloric sphincter, such that pyloric sleeve section (205), related to herein also as the pyloric sleeve region or first sleeve section, passes through the pyloric sphincter in a distal direction and remains therein. As detailed above, pyloric sleeve section (205) may be designed so that the pyloric sphincter is either capable of closing or remains, at least partially, opened. Duodenal sleeve section (209), related to herein also as the duodenal sleeve region or second sleeve section, stretches from the duodenal anchor (207) down the duodenum, at a predefined length, set as desired. According to some embodiments, the predefined length of duodenal sleeve section (209) is between about 27 cm and 112 cm.

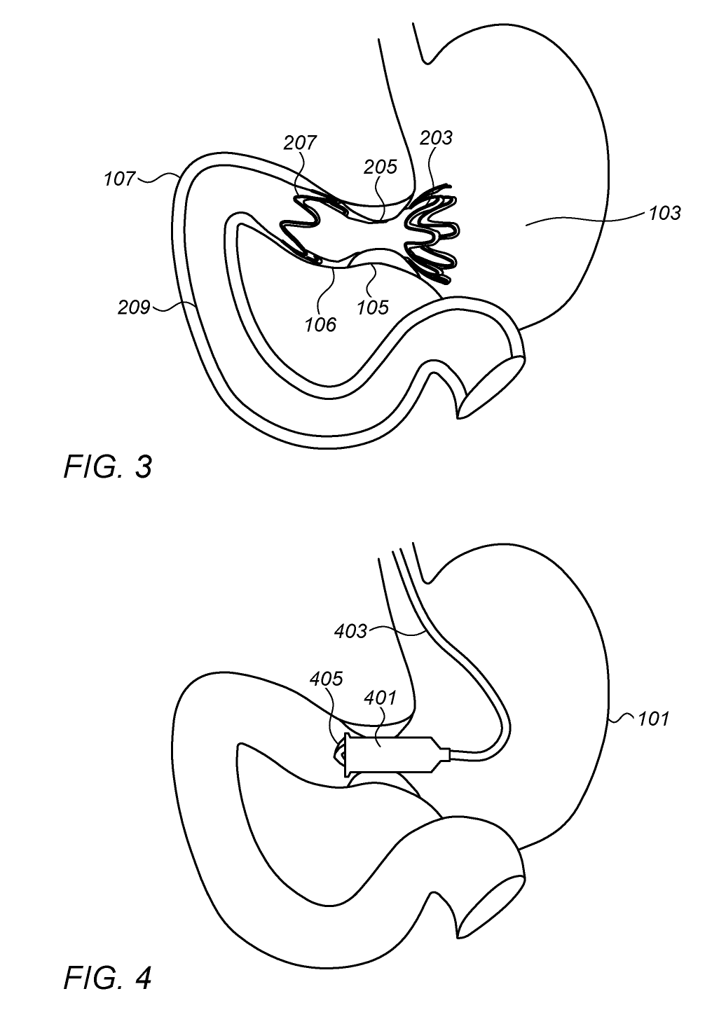

Reference is now made to FIG. 3, presenting a cross sectional view of an embodiment of the intragastric device of the invention after being deployed in the digestive tract. Gastric anchor (203) is anchored on the stomach side of pyloric sphincter (105), while duodenal anchor (207) is anchored on the duodenal side of the pyloric sphincter (105). According to some embodiments, the gastric anchor (203) is anchored in pylorus (103). According to some embodiments, the duodenal anchor (207) is anchored in the duodenal bulb (106). Pyloric sleeve section (205) passes through pyloric sphincter (105) and duodenal sleeve section (209) stretches from the duodenal anchor (207) down the duodenum (107).

Reference is now made to FIG. 4, presenting a cross sectional view of a portion of the digestive tract at the beginning of the deployment of an embodiment of the intragastric device. The intragastric device may be inserted transorally, wherein the device (not fully shown) is folded within capsule (401), which is attached to outer tube (403). Outer tube (403) extends from the mouth, through the oesophagus and into the stomach (101). Rounded element (405) may be in the form of a ring and may have both a folded and unfolded configuration, and may be partially exposed at the distal end of capsule (401), which presents the ring in the folded configuration.

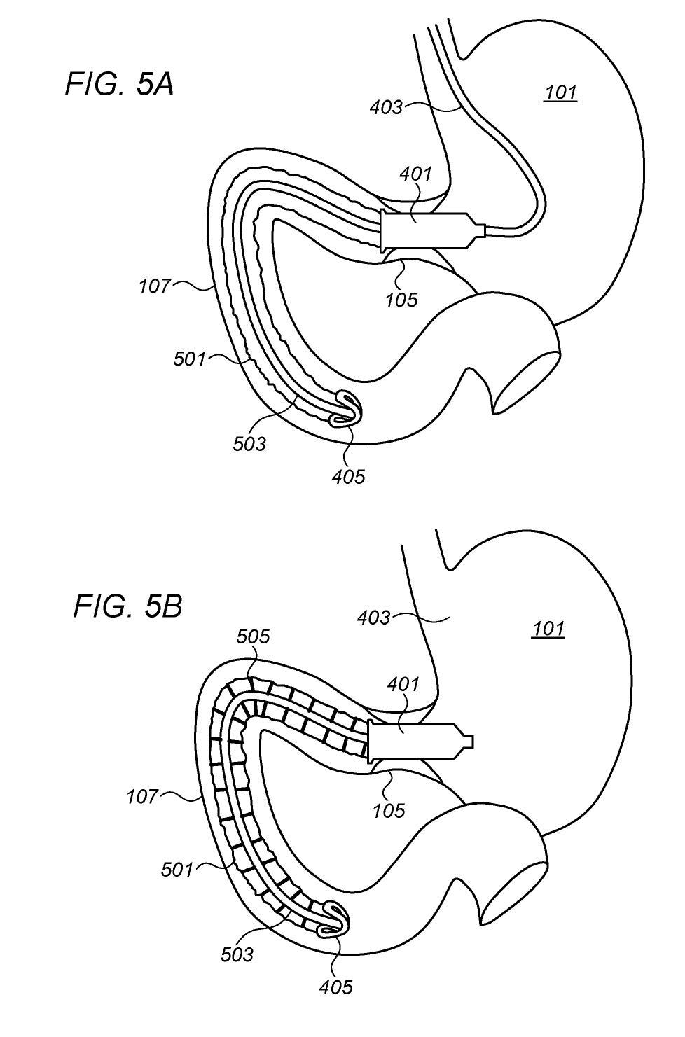

Reference is now made to FIG. 5, presenting a cross sectional view of a portion of the digestive tract during the deployment of an embodiment of the intragastric device. During the deployment of sleeve (501) (FIG. 5A), capsule (401) is positioned in the pyloric sphincter (105), and remains there while inner tube (503) extends distally along the duodenum (107). The distal end of inner tube (503) is coupled to rounded element (405), which, as presented, may be in the form of a ring having a folded configuration. Rounded element (405) is positioned at the distal end of sleeve (501). As detailed above, the rounded element (405) may be attached to sleeve (501) by any appropriate means. According to other embodiments, the rounded element (405) is a part of sleeve (501) and may be formed, e.g., by folding, rolling, of the distal end of sleeve (501), or any other appropriate means. By extending inner tube (503), sleeve (501) is deployed along the intestine. Rounded element (405) is pushed by inner tube (503), thereby spreading, or deploying, sleeve (501) along the intestine. Deployment of sleeve (501) gradually extends reinforcements (505) (FIG. 5B) along the length of sleeve (501) maintaining sleeve (501) open. Rounded element (405) could be made of similar to sleeve materials e.g., formed from a polymeric material as polyurethane, polyethylene, silicon, PTFE, not coated or coated with parylene. According to some embodiments, the sleeve could be formed of a bio-degradable material including materials that can be dissolved or liquefied by the gastric fluids or by specific fluids that will be used as part of the procedure. In order to reduce the rounded element size, in some examples the rounded element could be formed to reduce its size from an atraumatic end to a smaller size that will maintain its function as and have an end capable of supporting sleeve end.

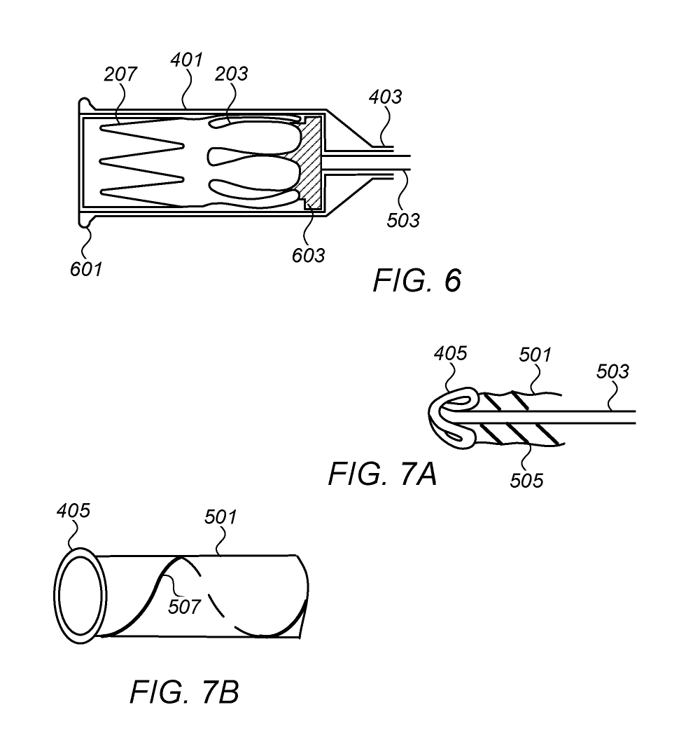

Reference is now made to FIG. 6, presenting a cross sectional view of an embodiment of the intragastric device compacted within capsule (401). The duodenal anchor (207) is in a folded/compacted configuration at the distal end of capsule (401) and gastric anchor (203) is at the proximal end of capsule (401). The distal end of the capsule (601) may be rounded to prevent tissue damage during deployment of the device. Pushing plate (603) may be placed at the proximal end of capsule (401), wherein gastric anchor (203) is compacted distally to pushing plate (603), such that during deployment, pushing plate (603) pushes against gastric anchor (203) and assists in the deployment of the device. Pushing plate (603) is attached to pushing wire not shown, which pushes pushing plate (603), is inside outer tube (403) and is pushed/fed in by the user from outside the mouth of the patient (not shown). It is noted that according to some embodiments, the tube related to herein, including the inner tube, may be replaced by any other appropriate means, such as a flexible wire protected by soft lining.

Reference is now made to FIGS. 7A and 7B, presenting a perspective view of an embodiment of the rounded element (405) in folded configuration (FIG. 7A) and in unfolded configuration (FIG. 7B). Rounded element (405), which is at the end of sleeve (501), is pushed by inner tube (503), thereby assisting in deploying sleeve (501) without damaging the tissue during deployment. Once inner tube (503) is removed by pulling it in the proximal direction (after the deployment of sleeve (501)), rounded element (405) is transformed from its folded configuration (FIG. 7A) to the unfolded configuration (FIG. 7B). Further, once deployed, rounded element (405) provides support to the distal end of sleeve (501) thus causing the distal end of the sleeve to remain opened. Reference numeral (505) and (507) mark sleeve 501 reinforcement elements

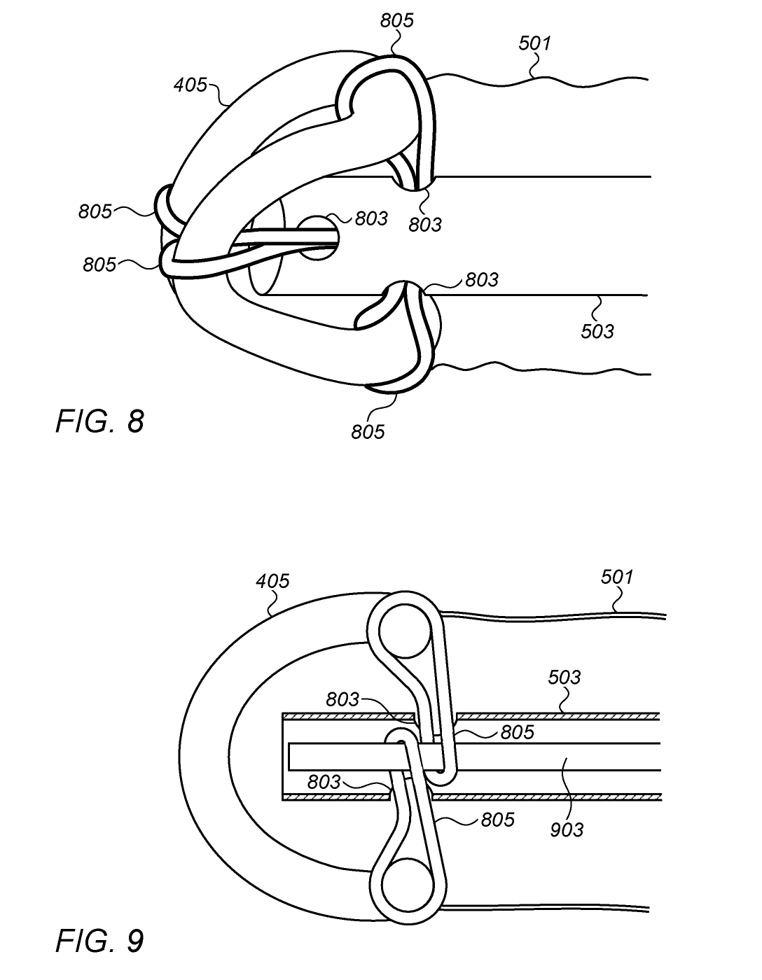

Reference is now made to FIG. 8, presenting a perspective view of an embodiment of rounded element (405), folded and attached to inner tube (503). During deployment of sleeve (501), rounded element (405) is folded to form a round shape, acting as a barrier between inner tube (503) and the body tissue, thereby preventing tissue damage during deployment. The size, shape, structure and/or configuration of rounded element (405) may also assist the deployment of sleeve (501) by peristaltic movements. Rounded element (405) may be attached to inner tube (503) by any appropriate connecting means, e.g., twine, guide wire and the like. As shown in FIG. 8, inner tube (503) may include any number of holes (803) through which connecting means (805) connect rounded element (405) to inner tube (503). It is noted that the connecting means may be configured in any number of loops around rounded element (405) or by any other appropriate means. Once sleeve (501) is deployed, connecting means (805) may be removed, severed or the like, after which inner tube (503) may be removed.

Reference is now made to FIG. 9, presenting a cross sectional view of an embodiment of the rounded element, folded and attached to the inner tube. As shown in FIG. 9, any number of connecting elements (805), connecting rounded element (405) to inner tube (503), may be coupled to wire (903) or to any other appropriate means, which is positioned inside inner tube (503). According to the presented embodiment, each connecting element, or section of connecting element, (805) exits inner tube (503) via holes (803) and then encircles rounded element (405). After the distal end of sleeve (501) is deployed as desired, wire (903) is pulled in the proximal direction, thereby loosening connecting elements (805). Thus, when pulling inner tube (503) in the proximal direction, connecting elements (805) completely pass through holes (803), allowing the inner tube to be removed, while rounded element (405) remains in place. Connecting elements may remain hanging from rounded element or be made from a biodegradable material and degrade in the body following sleeve deployment.

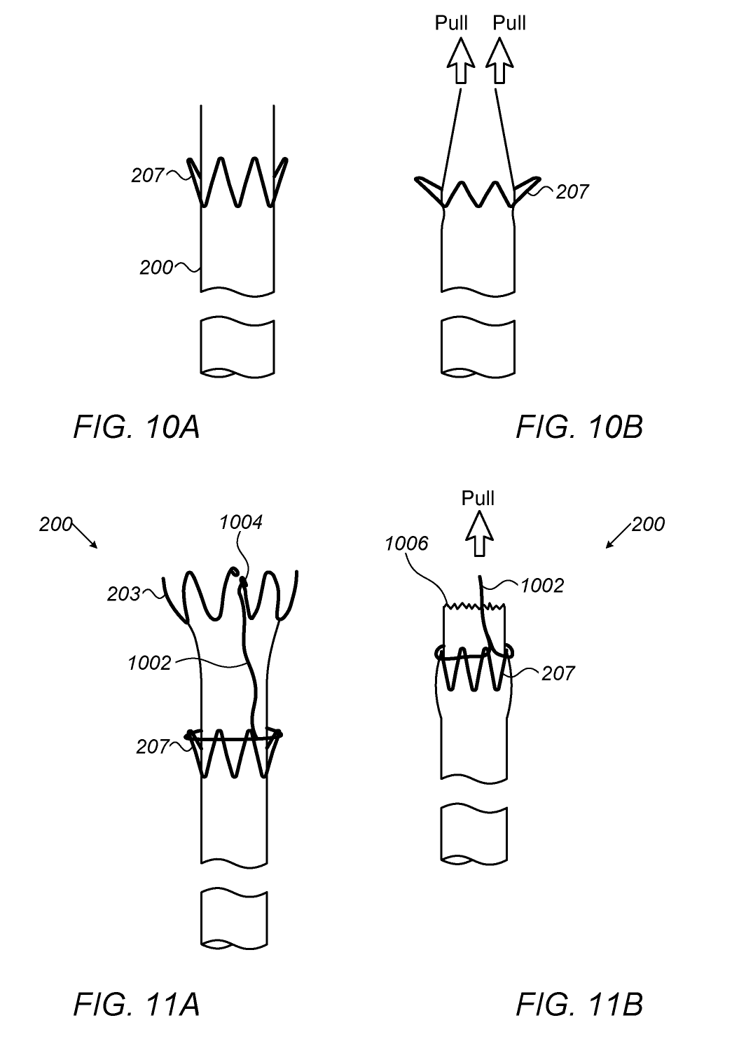

Reference is now made to FIGS. 10A and 10B, presenting an embodiment of the expandable duodenal anchor (207). As shown in FIGS. 10A and 10B, expandable duodenal anchor (207) is attached to device (200) (not fully shown), between pyloric sleeve section (205) and duodenal sleeve section (209) only at the distal end, such that FIG. 10A presents the shape of the anchor when no tension is applied to the sleeve, while FIG. 10B presents the shape of the anchor when proximal tension is applied on the sleeve. As shown in FIG. 10B, proximal tension causes expandable duodenal anchor (207) to be narrowed at the distal end and expanded at the proximal end, thus assisting in the resistance of the anchor to proximal movements, aiding in anchoring device (200) in the desired position.

Reference is now made to FIGS. 11A and 11B presenting proximal views of part of intragastric device (200), comprising gastric anchor (203), duodenal anchor (207), and coupling element (1002), wherein coupling element (1002) couples gastric anchor (203) to duodenal anchor (207). As shown in FIG. 11A, coupling element (1002) is attached on to one proximal end (1004) of gastric anchor (203), stretching axially down device (200) to duodenal anchor (207), to which it is attached at the proximal end, around the circumference of the anchor. According to some embodiments, any one of the ends of the anchor, e.g., proximal end of gastric anchor (1004), may be in the shape of a hole, hook, or the like, enabling the coupling element (1002) to easily be attached thereto and detached therefrom. Any such holes and/or hook may be found on the duodenal anchor (207) as well (not shown), through which the coupling element (1002) may be attached.

When coupling element (1002) is pulled in the proximal direction, gastric anchor (203) is collapsed (or transformed to a more linear shape, not shown) and device (200) is torn/severed along line (1006), distally to where the gastric anchor (203) was previously attached to device (200). Further pulling coupling element (1002) in the proximal direction causes duodenal anchor (207) to collapse, as shown in FIG. 11B.

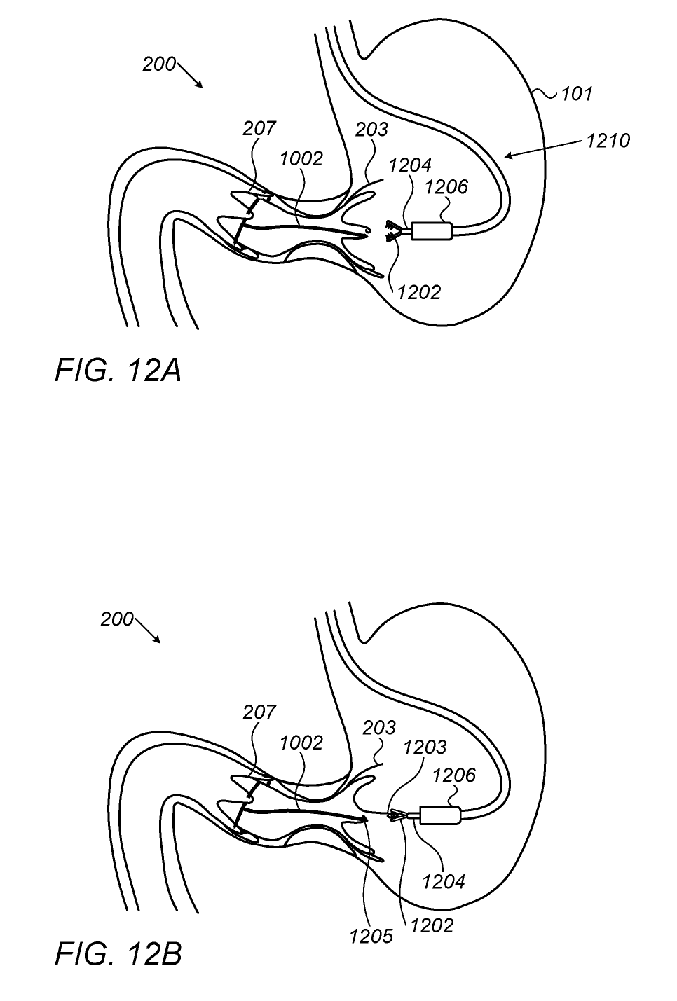

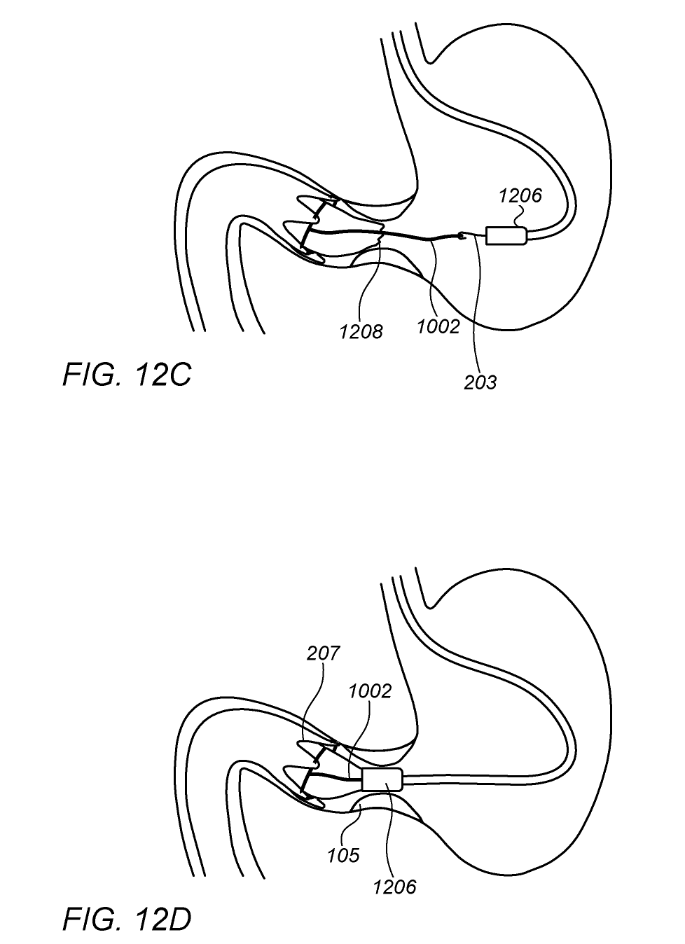

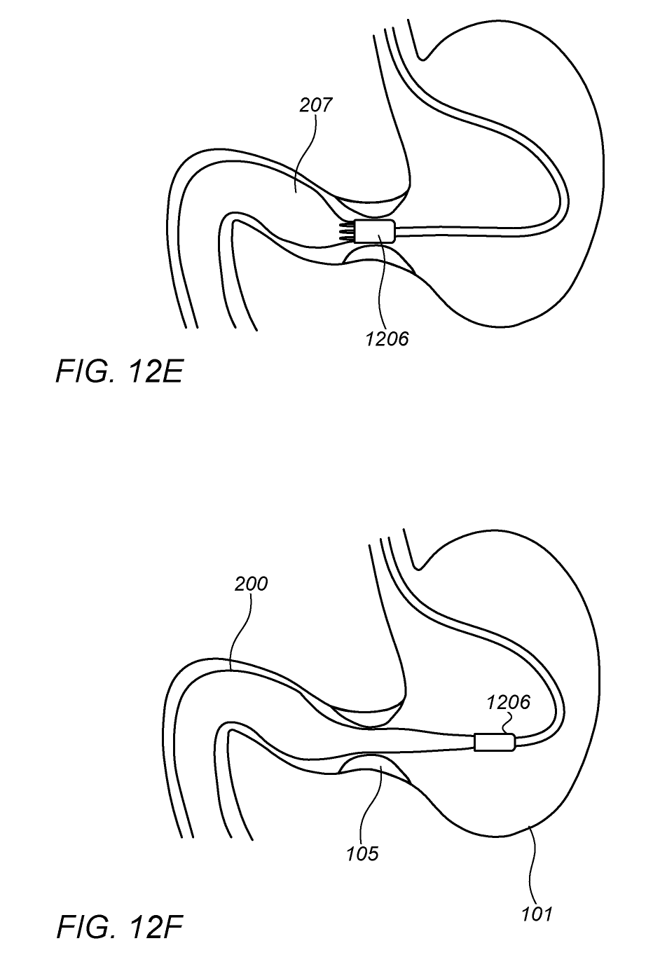

Reference is now made to FIG. 12A, presenting a cross sectional view of withdrawing device (1210) in stomach (101), wherein withdrawing device (1210) comprises grasping element (1202), inner sheath (1204) and outer sheath (1206). Shown also is intragastric device (200), comprising gastric anchor (203), having an opened structure, expandable duodenal anchor (207), attached to device (200) only at the distal end of anchor (207), and coupling element (1002), coupling gastric anchor (203) and duodenal anchor (207) to one another.

Reference is now made to FIG. 12B, presenting a cross sectional view showing grasping element (1202) that is grasping the end (1203) of gastric anchor (203). It is noted that one end (1203) of gastric anchor (203) is free, while other end (1205) is coupled, via coupling element (1002) to duodenal anchor (207). As shown in FIGS. 12B and 12C, once grasped, pulling inner sheath (1204) in the proximal direction into outer sheath (1206), pulls and collapses gastric anchor (203) and causes the detachment thereof from device (200). The detachment of gastric anchor (203) from device (200) creates tear line (1208). According to some embodiments, in order for gastric anchor (203) to be easily detached from device (200), device (200) may be weakened in the area of tear line (1208), e.g., by a perforation line or any other appropriate means. It is noted that FIG. 12C shows only the end of gastric anchor (203), wherein the rest of gastric anchor (203) is already pulled into outer sheath (1206) after assuming a more linear shape once detached from device (200).

After gastric anchor (203) is pulled (or folded or extended) into outer sheath (1206) and tear line (1208) is formed, as shown in FIG. 12C, the distal end of outer sheath (1206) is positioned inside pyloric sphincter (105), where it is attached via coupling element (1002) to duodenal anchor (207), as shown in FIG. 12D. By further pulling the inner sheath in the proximal direction (not shown), duodenal anchor (207) is contracted/collapsed/compacted and pulled into outer sheath (1206), as presented in FIG. 12E. This is possible since connecting element (1002) is connected to the circumference of duodenal anchor (207) and therefore, pulling on the connecting element (1002) causes the proximal end of duodenal anchor (207) to contract/collapse/be compacted. According to other embodiments, the connecting element (1002) may be attached to any additional means on duodenal anchor (207), wherein when connecting element (1002) is pulled in the proximal direction, those means cause the duodenal anchor (207) to contract. After duodenal anchor (207) is pulled into outer sheath (1206), outer sheath (1206) is pulled in the proximal direction out of pyloric sphincter (105) into stomach (101), followed by the remaining segment of device (200), as shown in FIG. 12F. The withdrawing device is removed from the stomach (101) of patient's body together with the remaining part of device (200).

Various aspects of the invention were described in greater detail in the different examples, which represent embodiments of this invention, and are by no means to be interpreted as limiting the scope of this invention.

What is claimed is:

1. An intragastric device (200) comprising a sleeve (501) including a pyloric sleeve region (205) and a duodenal sleeve region (209), a rounded element (405) at a distal end of the duodenal sleeve region (209) and an expandable duodenal anchor (207), which is positioned between the pyloric sleeve region (205) and the duodenal sleeve region (209).

2. The intragastric device (200) according to claim 1, wherein the sleeve (501) further comprises one or more additional anchors.

3. The intragastric device (200) according to claim 1, wherein the sleeve (501) is axially collapsible.

4. The intragastric device (200) according to claim 1, wherein the sleeve (501) includes reinforcements.

5. The intragastric device (200) according to claim 1, wherein the sleeve (501) resists structural degradation for at least six months within a gastrointestinal tract.

6. The intragastric device (200) according to claim 2, wherein the sleeve (501) further comprises a gastric anchor (203).

7. The intragastric device (200) according to claim 1, wherein only the distal end of the duodenal anchor (207) is attached to the sleeve (501).

8. The intragastric device (200) according to claim 2, wherein none of the additional anchors include tissue piercing elements.

9. The intragastric device (200) according to claim 2, wherein at least one of the additional anchors is prepared from a nitinol structure compressed to be delivered transorally and expandable for being in contact with at least part of antrum area of a stomach or intestine, depending on the position of the additional anchor.

10. The intragastric device (200) according to claim 6, wherein the duodenal anchor (207) and the gastric anchor (203) are coupled by a coupling element (1002).

11. The intragastric device (200) according to claim 6, wherein the gastric anchor (203) is prepared as an opened shape, wherein at least two ends (1203, 1205) of the gastric anchor (203) are connected to one another only by a polymeric material.

12. The intragastric device (200) according to claim 11, wherein the polymeric material is the polymeric material of the sleeve (501).

13. The intragastric device (200) according to claim 11, wherein at least one of the ends (1205) of the gastric anchor (203) is coupled to the duodenal anchor (207) at a proximal end of the duodenal anchor (207).

14. The intragastric device (200) according to claim 1 for use in treating type II diabetes, small intestine perforation, small intestine abscess, obesity, or any combination thereof

15. Method of deploying the intragastric device (200) according to claim 1, said method comprising:
delivering the intragastric device (200) folded within a capsule (401) through mouth, wherein the capsule (401) is attached to an outer tube (403);
positioning the capsule (401) in a pyloric sphincter (105);
using an inner tube (503) connected to a rounded element (405) to push and thereby deploy the sleeve (501) from the capsule (401) positioned in the pyloric sphincter (105) along a predefined length of a small intestine (107), thereby releasing the intragastric device (200) from the capsule (401), thus causing the device (200) to be transformed from a compacted configuration to an expanded configuration, wherein a duodenal anchor (207) is positioned on a duodenal side of the pyloric sphincter (105);
removing the inner tube (503); and
pulling the outer tube (403) out of the mouth.

16. The method according to claim 15, wherein the rounded element (405) has a folded configuration and an unfolded configuration and wherein the rounded element (405) is in the folded configuration when connected to the inner tube (503) and is further transformed to the unfolded configuration when the inner tube (503) is removed.

17. Method of removing the intragastric device (200) according to claim 13 from a patient's body, said method comprising:
introducing a withdrawing device (1210) into a patient's stomach (101), wherein the withdrawing device (1210) comprises an outer sheath (1206), an inner sheath (1204), positioned inside the outer sheath (1206), and a grasping element (1202) extending from the distal end of the inner sheath (1204);
grasping an end (1202) of the gastric anchor (203) that is not directly attached to a coupling element (1002);
pulling the inner sheath (1204) in a proximal direction until the gastric anchor (203) is pulled into the outer sheath (1206) and the sleeve (501) is torn distally to where the gastric anchor (203) was attached to a sheet (501), such that a coupling element (1002) provides a connection between t a duodenal anchor (207) and the withdrawing device (1210);
positioning a distal end of the outer sheath (1206) in a pyloric sphincter (105);
further pulling the inner sheath (1204) in the proximal direction, which pulls the coupling element (1002), causing the duodenal anchor (207) to contract and to be pulled into the outer sheath (1206); and
pulling the outer sheath (1206) in a proximal direction from the pyloric sphincter (105) through the stomach (101) and out of the patient's body.

18. A withdrawing device (1210) for withdrawing an intragastric device comprising an outer sheath (1206), an inner sheath (1204), positioned inside the outer sheath (1206), and a grasping element (1202) extending from the distal end of the inner sheath (1204).

19. A kit comprising the intragastric device (200) according to claim 13 and the withdrawing device (1210) according to claim 18.

20. The intragastric device (200) according to claim 4, wherein the reinforcements are made from at least one of a group of materials consisting of nitinol, polymer such as polyurethane, polyethylene, silicon, PTFE or from composite material or by variation in a sleeve polymer thickness.
